SEQUENCE LISTING (24 sequences)

SEQ ID NO: 1 - protein, 9 aa
ASNENMDAM

SEQ ID NO: 2 - protein, 10 aa
DTPLIPLTIF

SEQ ID NO: 3 - protein, 9 aa
EEGAIVGEI

SEQ ID NO: 4 - protein, 10 aa
EENLLDFVRF

SEQ ID NO: 5 - protein, 9 aa
FAPGNYPAL

SEQ ID NO: 6 - protein, 9 aa
FLRGRAYGL

SEQ ID NO: 7 - protein, 9 aa
HLAAQGMAY

SEQ ID NO: 8 - protein, 9 aa
IVTDFSVIK

SEQ ID NO: 9 - protein, 9 aa
KEHVIQNAF

SEQ ID NO: 10 - protein, 97 aa
MSTNSFLRGRAYGLQAKWRLQTLEENLLDFVRFSVRDRLARLKEHVIQNAFYPLHEQHGMHLAAQGMAYDTPLIPLTIFIVTDFSVIKNNLVSGPEH

SEQ ID NO: 11 - protein, 9 aa
QAKWRLQTL

SEQ ID NO: 12 - protein, 9 aa
RPQASGVYM

SEQ ID NO: 13 - protein, 8 aa
SDYEGRLI

SEQ ID NO: 14 - protein, 10 aa
SGPSNTPPEI

SEQ ID NO: 15 - protein, 9 aa
SIINFEEKL

SEQ ID NO: 16 - protein, 8 aa
SIINFEKL

SEQ ID NO: 17 - protein, 9 aa
SVRDRLARL

SEQ ID NO: 18 - protein, 9 aa
SYIPSAEKI

SEQ ID NO: 19 - protein, 8 aa
TYQRTRAL

SEQ ID NO: 20 - protein, 9 aa
TYQRTRALV

SEQ ID NO: 21 - protein, 9 aa
YPHFMPTNL

SEQ ID NO: 22 - protein, 9 aa
YPLHEQHGM

SEQ ID NO: 23 - protein, 107 aa
MSRASNENMDAMSIINFEKLTYQRTRALVSDYEGRLIYPHFMPTNLTSSGPSNTPPEIFAPGNYPALSYIPSAEKIEEGAIVGEIRPQASGVYMPRNNLVSGPEHLZ

SEQ ID NO: 24 - DNA, 327 bp; single-stranded, linear
atgtctagagccagcaacgagaacatggacgccatgagcatcatcaacttcgagaagcttacctaccagaggaccagggccctggtgagcgactacgagggcaggctgatctacccacacttcatgcctaccaacctgactagtagtgggcccagtaacaccccgccagagatcttcgccccaggcaactaccctgccctgagctacatcccaagcgccgagaagatcgagagaggcgccatcgtgggcgagatcaggcctcaggccagcggcgtgtacatgcctaggaacaacctggtgtccggccccgaggacttataggtcgac

(19) United States
(12) Patent Application Publication
(10) Pub. No.: US 2007/0172461 A1
(43) Pub. Date: Jul. 26, 2007

(54) Polyepitope vaccines

(75) Inventors: Andreas Suhrbier, Newmarket (AU); Scott Anthony Thomson, Ferny Hills (AU); Rajiv Khanna, Herston (AU); Scott Renton Burrows, Bald Hills (AU); Barbara Elizabeth Howieson Coupar, East Geelong (AU); Denis James Moss, Arana Hills (AU)

Correspondence Address: Steven L. Highlander, Esq.;FULBRIGHT & JAWORSKI L.L.P., 600 Congress Avenue, Suite 2400, Austin, TX, 78701, US

(73) Assignees: The Council of the Queensland Institute of Medical Research; Commonwealth Scientific and Industrial Research Organisation; The University of Melbourne; The walter and Eliza Hall Institute of Medical Research of Royal Melbourne Hospital; Biotech Australia PTY Limited; CSL Limited

(21) Appl. No.: 10/884,183

(22) Filed: Jul. 2, 2004

Related U.S. Application Data
(63) Continuation of application No. 09/576,101, filed on May 22, 2000, now abandoned.
Application No. 09/576,101 is a continuation of application No. 08/776,337, filed on Apr. 21, 1997.

(30) Foreign Application Priority Data
Jul. 27, 1994 (AU) PM 7079
Feb. 8, 1995 (AT) PN 1009

(51) Int. Cl. A61K 48/00; A61K 39/275; A61K 39/285; A61K 39/02

(52) U.S. Cl. 424/93.2; 514/44; 424/232.1; 424/234.1; 977/802

(57) ABSTRACT

The present invention relates to a recombinant polyepitope cytotoxic T lymphocyte vaccine. The vaccine comprises at least one recombinant protein including a plurality of cytotoxic T lymphocyte epitopes from one or more pathogens, wherein the at least one recombinant protein is substantially free of sequences naturally found to flank the cytotoxic T lymphocyte epitopes. In addition the present invention also provides a polynucleotide including at least one sequence encoding a plurality of cytotoxic T lymphocyte epitopes from one or more pathogens.

The present invention relates to vaccines containing a plurality of cytotoxic T lymphocyte epitopes and to polynucleotides including sequences encoding a plurality of cytotoxic T lymphocyte epitopes.

CD8+αβ cytotoxic T lymphocytes (CTL) recognise short peptides (epitopes, usually 8-10 amino acids long) associated with specific alleles of the class I major histocompatability complex¹ (MHC). The peptide epitopes are mainly generated from cytosolic proteins by proteolysis, a process believed to involve the multicatalytic proteosome complex²⁻⁷. Peptides of appropriate length are transported into the endoplasmic reticulum where specific epitopes associate with MHC. The MHC/epitope complex is then transported to the cell surface for recognition by CTL. The influence of sequences flanking CTL epitopes on the proteolytic processing of these epitopes remains controversial⁸⁻¹². However, by constructing a recombinant vaccinia coding for an artificial polypeptide protein containing nine CD8 CTL epitopes in sequence, the present inventors have determined that the natural flanking sequences of CTL epitopes are not required for class I processing, that is each epitope within the polyepitope protein was always efficiently processed and presented to appropriate CTL clones by autologous polyepitope vaccinia infected target cells.

Accordingly, in a first aspect, the present invention consists in a recombinant polyepitope cytotoxic T lymphocyte vaccine, the vaccine comprising at least one recombinant protein including a plurality of cytotoxic T lymphocyte epitopes from one or more pathogens, wherein the at least one recombinant protein is substantially free of sequences naturally found to flank the cytotoxic T lymphocyte epitopes.

Preferably, the at least one recombinant protein does not include any sequences naturally found to flank the cytotoxic T lymphocyte epitopes. However, it should be understood that small lengths (e.g. 1-5 amino acids) of sequences naturally found to flank the cytotoxic T lymphocyte epitopes may be included. The phrase “substantially free of sequences naturally found to flank the cytotoxic T lymphocyte epitopes” is to be taken as including such short lengths of flanking sequences.

In a second aspect, the present invention consists in a polynucleotide, the polynucleotide including at least one sequence encoding a plurality of cytotoxic T lymphocyte epitopes from one or more pathogens, and wherein the at least one sequence is substantially free of sequences encoding peptide sequences naturally found to flank the cytotoxic T lymphocyte epitopes.

Again, it is to be understood that “substantially free of sequences encoding peptide sequences naturally found to flank the cytotoxic T lymphocyte epitopes” includes the possibility of including short peptide (e.g 1-5 amino acids) sequences naturally found to flank the cytotoxic T lymphocyte epitopes.

In a third aspect, the present invention consists in a nucleic acid vaccine, the vaccine comprising the polynucleotide of the second aspect of the present invention and an acceptable carrier.

In a fourth aspect, the present invention consists in a vaccine formulation, the vaccine comprising the recombinant protein of the first aspect of the present invention and an acceptable carrier and/or adjuvant.

In a preferred embodiment of the present invention the at least one recombinant protein includes, and the at least one sequence encodes, at least three cytotoxic T lymphocyte epitopes.

In a further preferred embodiment, the epitopes are from multiple pathogens.

It is also envisaged that the vaccines according to the invention may include immunomodulatory compounds (such as cytokines), other proteins/compounds (such as melittin or regulatory proteins) and/or adjuvants. The vaccines may also include helper epitopes/CD4 epitopes and proteins, B-cell epitopes or proteins containing such epitopes, for example tetanus toxoid. Another example of a vaccine according to the invention comprises a recombinant vaccine construct wherein the polytope including the CTL epitopes is linked to an extracellular glycoprotein or glycoproteins containing B-cell and/or CD4 epitopes.

The vaccines according to the invention may be delivered by various vectors, for example vaccinia vectors, avipox virus vectors, bacterial vectors, virus-like particles (VLP's) and rhabdovirus vectors or by nucleic acid vaccination technology. As polytope proteins are likely to be sensitive to proteolysis during manufacture and/or serum following injection, we envisage that such vaccines may best be delivered using nucleic acid vaccination technologies¹², vector systems or adjuvant systems which protect the polytope protein from proteolysis. Additional information regarding vectors may be found in Chatfield et al, Vaccine 7, 495-498, 1989; Taylor et al, Vaccine 13, 539-549, 1995; Hodgson “Bacterial Vaccine Vectors” in Vaccines in Agriculture.

A polytope vaccine according to the invention may also include a large number of epitopes (e.g. up to 10 or more) from one pathogen so that the HLA diversity of the target population is covered. For instance a vaccine containing epitopes restricted by HLA A1, A2, A3, A11 and A24 would cover about 90% of the Caucasian population.

A polytope vaccine according to the invention may also be constructed such that the multiple epitopes are restricted by a single HLA allele.

In a preferred embodiment of the fourth aspect of the present invention the vaccine formulation includes ISCOMs. Information regarding ISCOMs can be found in Australian patent No 558258, EP 019942, US4578269 and US4744983, the disclosures of which are incorporated herein by reference.

In order that the nature of the present invention may be more clearly understood preferred forms thereof will now be described with reference to the following examples and accompanying Figures in which:—

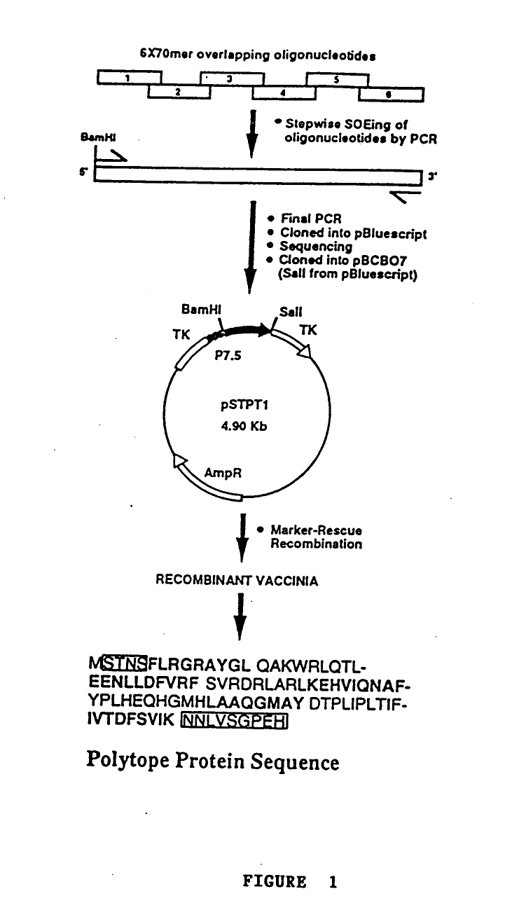

FIG. 1. Construction of a recombinant vaccinia that expresses a synthetic DNA insert coding for the polytope protein, which contains nine CTL epitopes in sequence. Boxed sequences representing linear B cell epitopes.

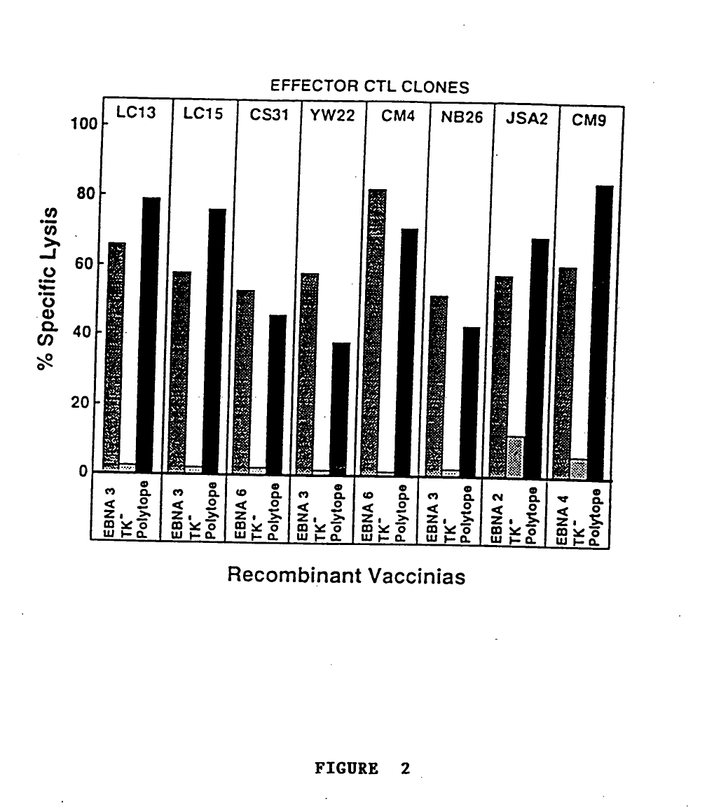

FIG. 2. CTL recognition of each epitope expressed in the recombinant polytope vaccinia construct.

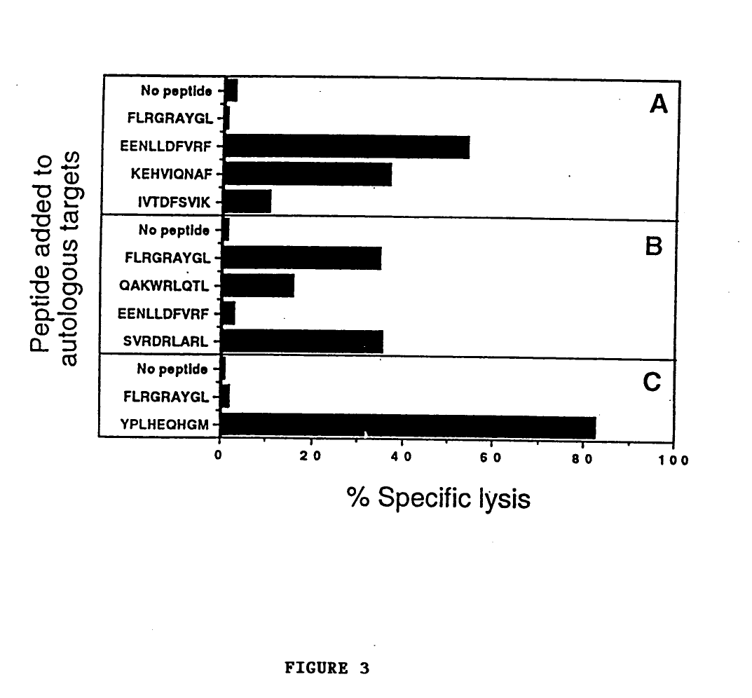

FIG. 3. Polytope vaccinia can recall epitope specific responses. Bulk effectors from donors (A) CM-A24, A11, B7, B44; (B) YW-A2, B8, B38 and (C) NB-A2, A24, B7, B35 were generated by infecting peripheral blood mononuclear cells (PBMC) with the polytope vaccinia at a MOI of 0.01 for 2 hours followed by 2 washes. After 10 days culture in 10% FCS/1640 RPMI the bulk effectors were used against autologous phytohaemagglutinin T cell blasts target cells (E:T 20:1) sensitised with the indicated peptide (10 μM) in a standard 5 hour chromium release assay¹⁴. Bulk effectors generated by the addition of irradiated autologous A type LCLs¹⁴ (LCL to PBMC ratio 1:50) gave similar results to that shown above.

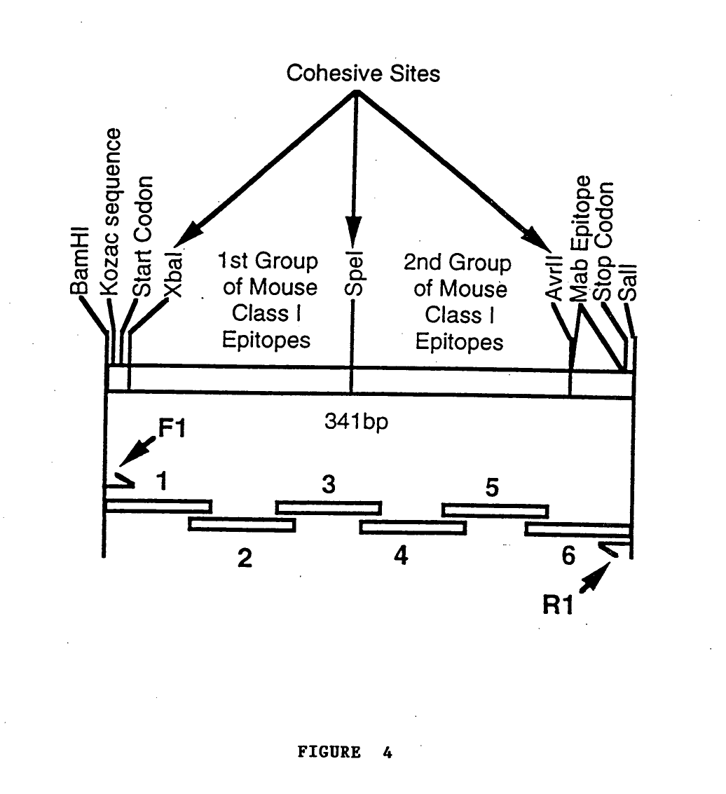

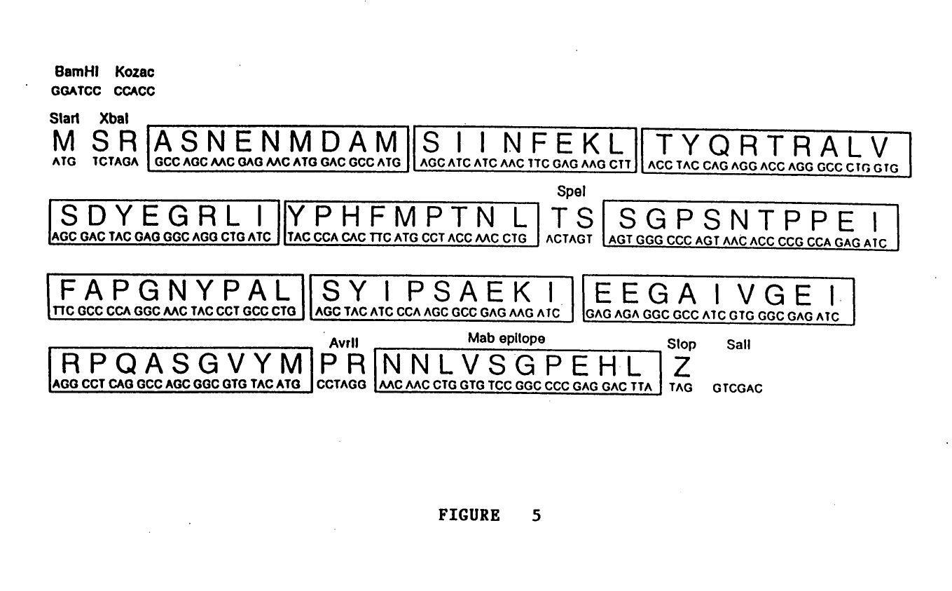

FIG. 4 shows the construction of a polytope DNA insert including ten murine CTL epitopes.

FIG. 5 shows the sequence of the polytope DNA insert of FIG. 1.

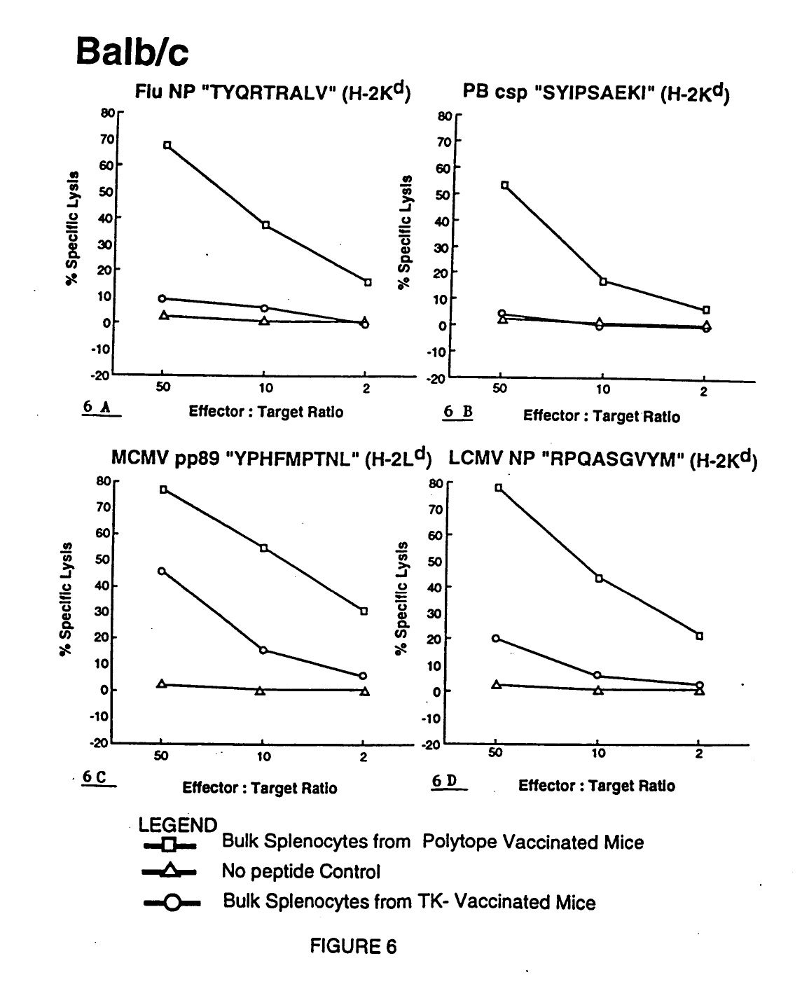

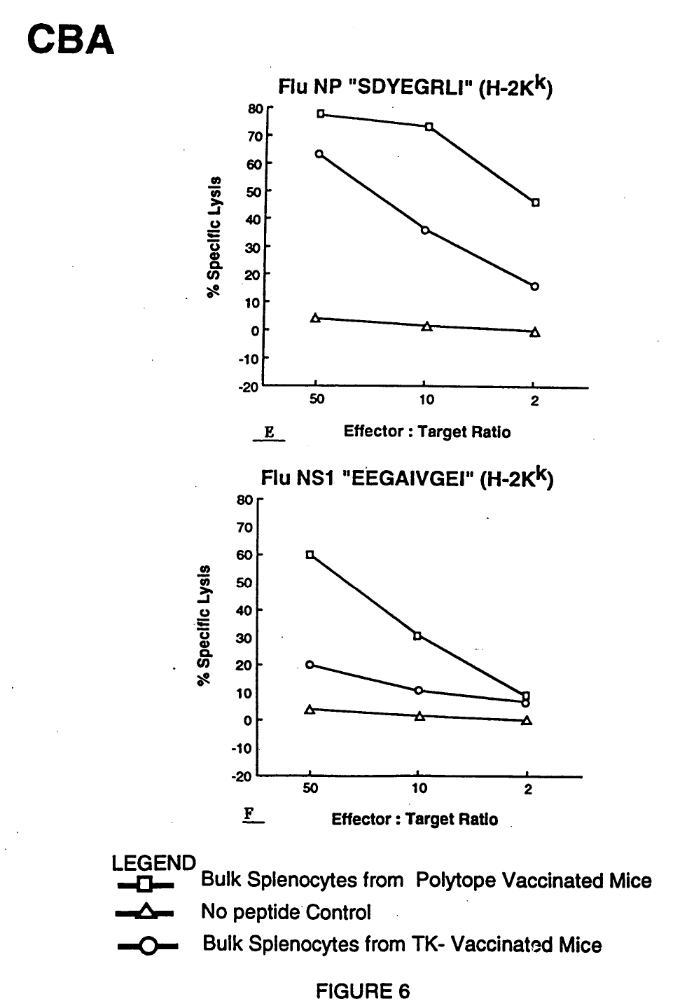

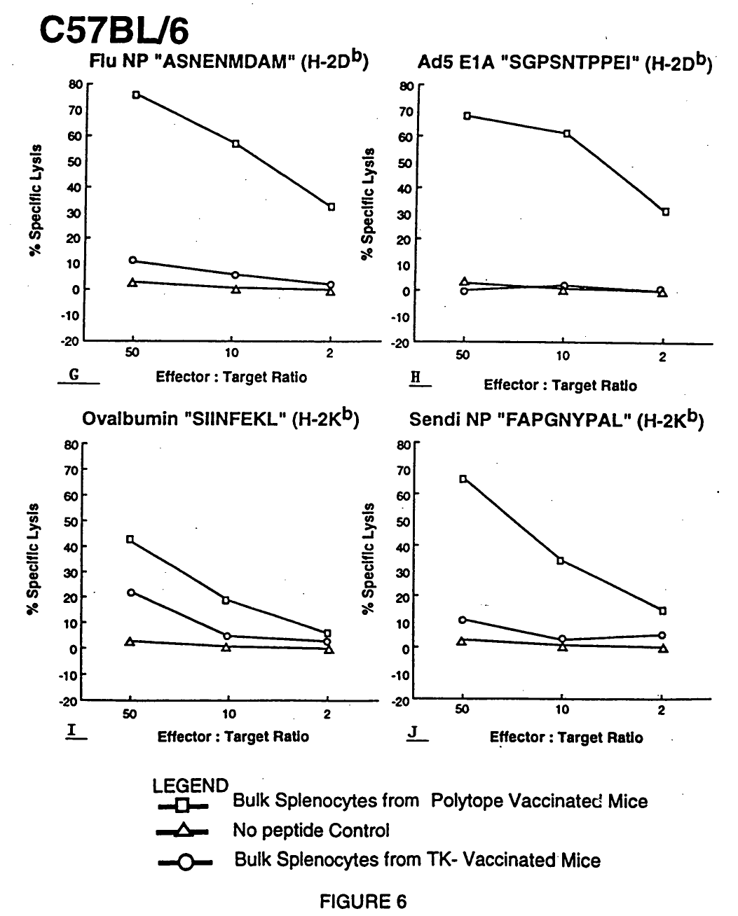

FIG. 6 provides results of CTL assays conducted on splenocytes harvested from mice vaccinated with recombinant vaccinia including the DNA insert of FIG. 3.

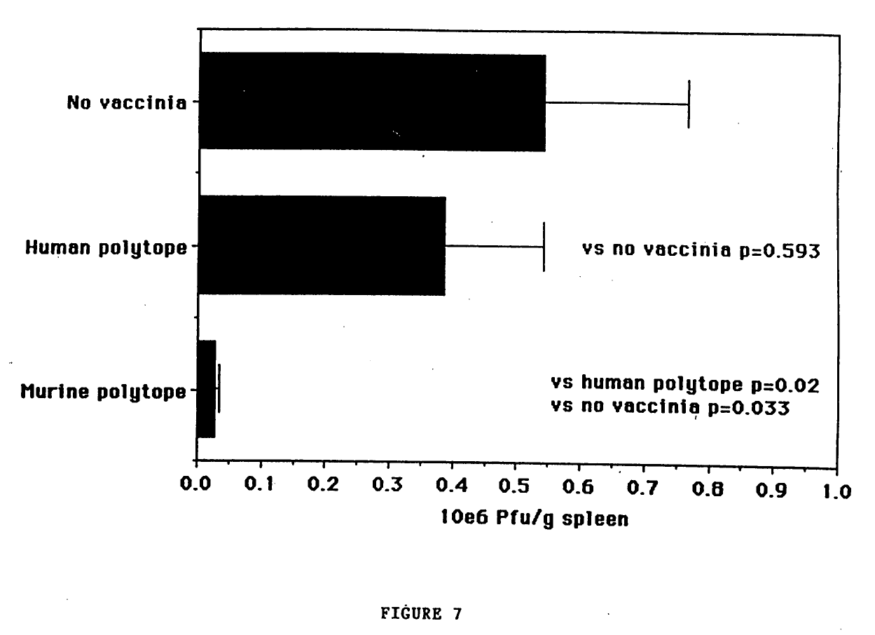

FIG. 7 shows comparison of spleen MCMV titres (±standard error) 5 weeks after polytope vaccinia vaccination and 4 days after MCMV challenge. P values—unpaired student t-test

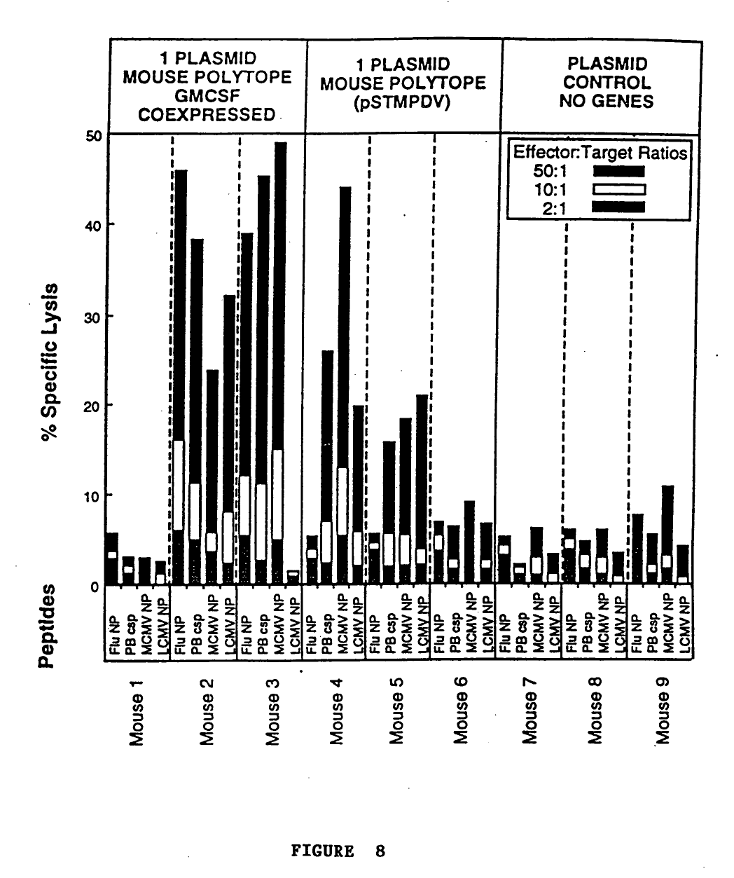

FIG. 8 DNA vaccination with different plasmids in Balb/c mice.

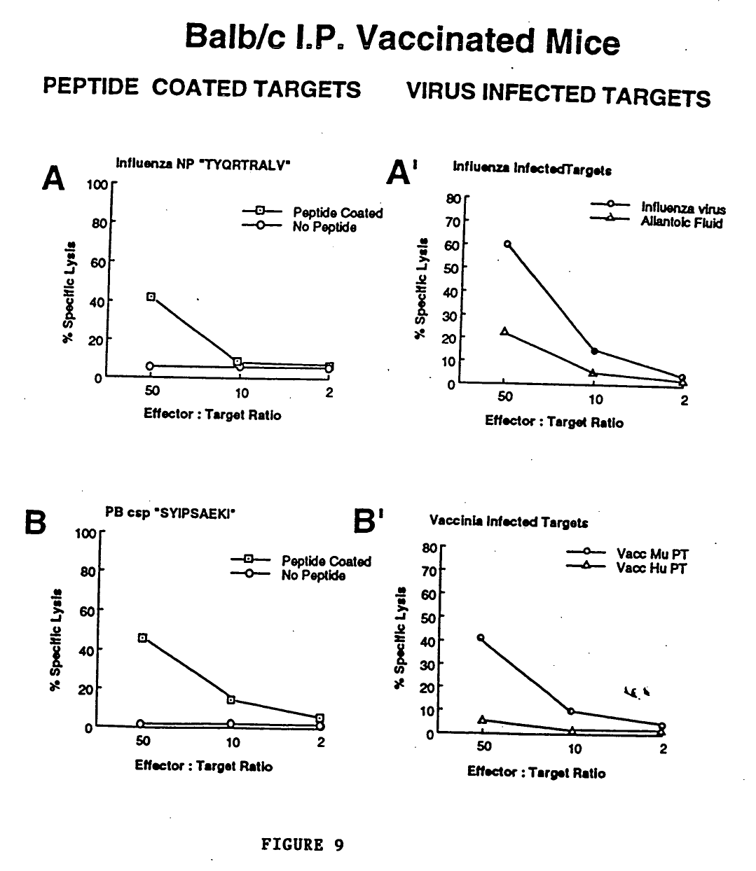

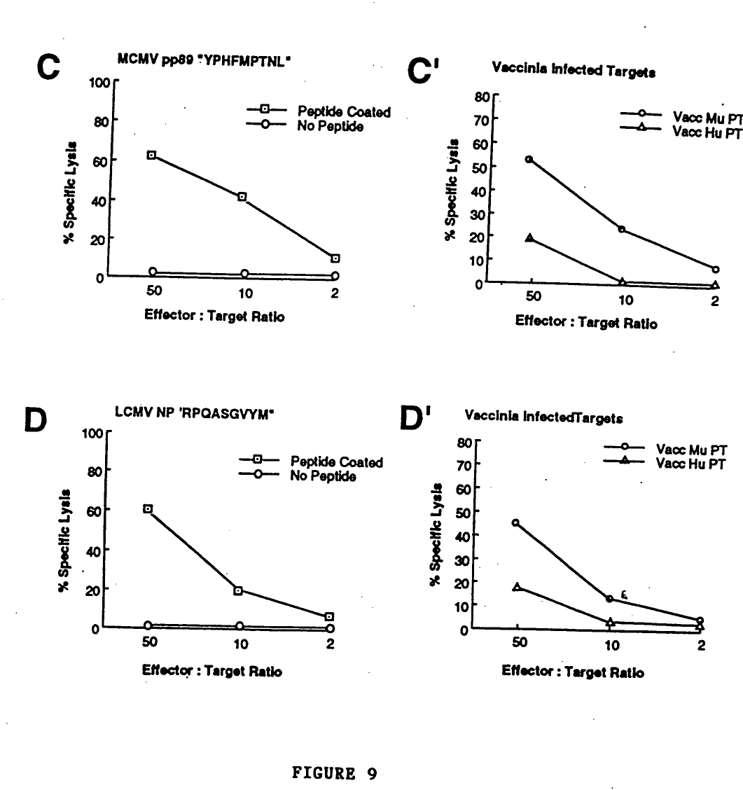

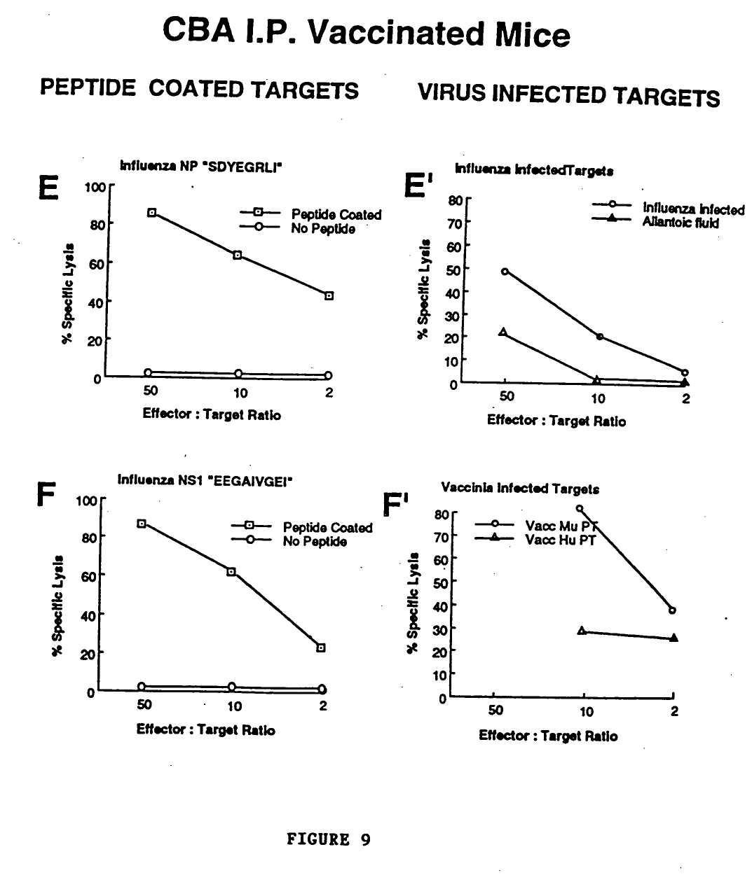

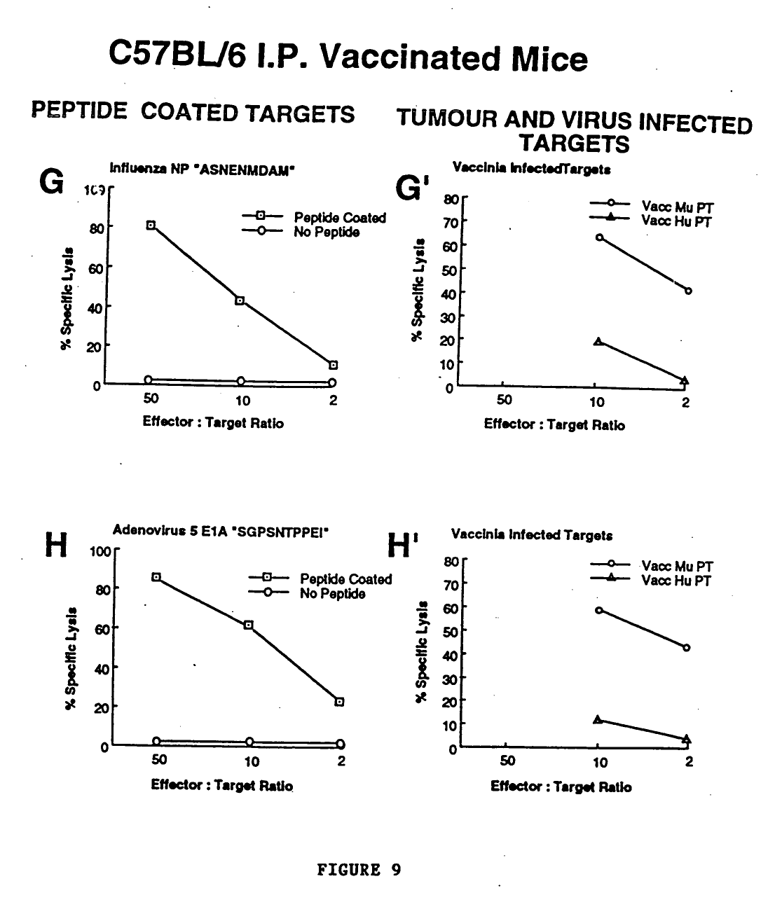

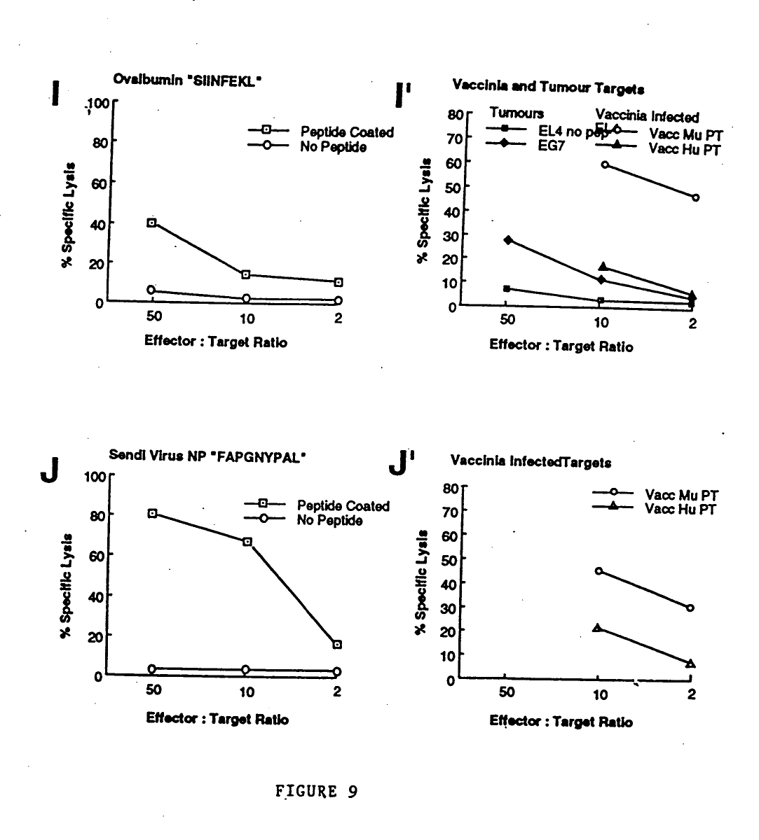

FIG. 9. Murine Polytope vaccinia immunised (IP) mice from these strains (Balb/c, CBA, C56BL/6) had the spleens removed and splenocytes restimulated with peptide (eg for A and A′). effectors were generated by stimulation with influenza NP peptide “TYQRTRALV”). The effectors were then used on peptide coated targets (A-J), virus infected targets (A′-J′) and tumour targets (I′). Virus infected targets were either infected (A′, F′), with allantoic fluid as negative control or murine polytope vaccinia (Vacc Mu PT) (B′-D′, F′-J′), with human polytope vaccinia (Vacc Hu PT) as the negative control.

### EXAMPLE 1

Nine, class I restricted, CTL epitopes from several Epstein-Barr virus nuclear antigens (EBNA) have previously been defined using CTL clones^{10, 18-20}. The clones were isolated from a panel of normal healthy Epstein-Barr virus (EBV) seropositive donors and were restricted by different HLA alleles (Table 1). A recombinant polyepitope vaccinia (polytope vaccinia), which coded for a single artificial protein containing all nine of these CTL epitopes, was constructed (see FIG. 1). The DNA sequence coding for this protein was made using splicing by overlap extension (SOEing) and the polymerase chain reaction (PCR) to join six overlapping oligonucleotides. The insert was cloned into pBluescript II, checked by sequencing and transferred into pBCBO7¹⁵ behind a vaccinia promoter to generate pSTPT1. This plasmid was then used to generate the polytope vaccinia virus by marker-rescue recombination¹⁶ The artificial polytope protein expressed by this vaccinia therefore containing no sequences naturally found to flank the CTL epitopes in their proteins of origin (FIG. 1).

TABLE 1
Description of the CTL clones, their cognate
epitopes, the proteins of origin (source) and their HLA
restriction.

| | COGNATE | | HLA | |
|---|---|---|---|---|
| CTL CLONES | EPITOPES | SOURCE | RESTRICTION | REFS |
| LC13 | FLRGRAYGL | EBNA3 | B8 | 13 |
| LC15 | QAKWRLQTL | EBNA3 | B8 | 14 |
| CS31 | EENLLDFVRF | EBNA6 | B44 | 15 |
| YW22 | SVRDRLARL | EBNA3 | A0203 | 14 |
| CM4 | KEHVIQNAF | EBNA6 | B44 | 13 |
| NB26 | YPLHEQHGM | EBNA3 | B3501 | 14 |
| LX1* | HLAAQGMAY | EBNA3 | ? | 14 |
| JSA2 | DTPLIPLTIF | EBNA3 | B51^{#}/A2 | 13 |
| CM9 | IVTDFSVIK | EBNA4 | A11 | 16 |

The first two letters of the clones refer to the donors.
*Not tested (see FIG. 2).
^{#}Recent evidence suggests this epitope may be restricted by HLA-B51. All the epitopes have been minimalised except EENLLDFVRF and DTPLIPLTIF.

A DNA sequence coding for the polytope amino acid sequence was designed with codons most frequently used in mammals and incorporated a Kozac sequence¹³ and a BamHI site upstream of the start codon. Six 70mer oligonucleotides overlapping by 20 base pairs were spliced together using Splicing by Overlap Extension (SOEing)¹⁴ in 20 μl reactions containing standard PCR buffer, 2 mM MgCl₂, 0.2 mM dNTPs, 1.5 U of Taq polymerase (hot start at 94° C.) using the following thermal program: \94° C. for 10 seconds, 45° C. for 20 seconds and 72° C. for 20 seconds (40 cycles). Half of each gel purified dimer sample was combined in a second PCR reaction (12 cycles) with the addition of 0.5 μl of α³²P dCTP. The reaction was run on a 6% acrylamide gel and a slice corresponding to the position of the hexamer product was isolated. Two 20mer oligonucleotides were used to PCR amplify the hexamer using an annealing temperature of 56° C. and 25 cycles. The gel purified fragment was cloned into the EcoRV site of pBluescript II KS-, was checked by sequencing and cloned behind the vaccinia P7.5 early/late promoter using the BamHI/SalI sites in the vaccinia plasmid vector pBCBO7¹⁵ to generate pSTPT1. Construction of a TK− recombinant virus was carried out using marker rescue combination between pSTPT1 and W-WR-L929 as described previously¹⁶. Plaque purified virus was tested for TK phenotype and for appropriate genome arrangement by Southern blotting of viral DNA¹⁶.

To establish whether each epitope could be efficiently processed from the polytope protein the polytope vaccinia was used to infect a panel of target cells, which expressed the HLA alleles restricting each epitope. Autologous CTL clones specific for each epitope were then used as effector cells in standard chromium release assays. In all cases tested the CTL clones recognised and killed the HLA matched target cell infected with the polytope vaccinia and the appropriate (see Table 1) EBNA vaccinia (positive controls), but not the TK-vaccinia (negative controls) (FIG. 2).

FIG. 2 shows CTL recognition of each epitope expressed in the polytope vaccinia construct. Effector CTL are listed in Table 1 (E:T ratio 5:1). Target cells (see below) were infected with recombinant vaccinia expressing either (i) the EPV nuclear antigen (EBNA) recognised by the CTL clone (see Table 1) (positive control), (ii) TK− (negative control), or (iii) the polytope construct (i.e., Polytope vaccinia). Vaccinia infection of the target cells was at a multiplicity of infection of 5:1 followed by 14-16 hour incubation at 37° C. prior to use in the standard, 5 hour, ⁵¹Cr-release assay²⁹. Clone LX1 was no longer available at the time of assay. Target cells; there are two types of EBV, A and B-type, whose EBNA protein sequences differ significantly. CTL clones LC13, LC15, CM4, NB26, JSA2 and CM9 recognise cells transformed with A-type EBV but not B-type EBV, and CTL clones CS31 and YW22 recognise cells transformed with A-type EBV and EBV¹⁰, 18-20. The target cells used for the A-type specific CTL were therefore autologous lymphoblastoid cell lines transformed with B-type virus (B-type LCLS). The target cell for CS31 and YW22 were EBV negative B cell blasts, established using anti-CD40 antibody and rIL-4²¹.

An additional series of experiments used the polytope vaccinia to stimulate in vitro a secondary CTL response from peripheral blood mononuclear cells (PBMC) obtained from healthy EBV seropositive donors. The resulting bulk CTL cultures were then used as effectors against peptide epitope sensitised autologous PHA blasts in a standard chromium release assay. The polytope vaccinia was capable of recalling CTL responses which were specific for epitopes restricted by the HLA alleles expressed by each donor (FIG. 3).

FIG. 3 shows that polytope vaccinia can recall epitope specific responses. Bulk effectors from donors (A) CM-A24, A11, B7, B44; (B) YW-A2, B8, B38 and (C) NB-A2, A24, B7, B35 were generated by infecting peripheral blood mononuclear cells (PBMC) with the polytope vaccinia at a MOI of 0.01 for 2 hours followed by 2 washes. After 10 days culture in 10% FCS/1640 RPMI the bulk effectors were used against autologous phytohaemagglutinin T cell blasts target cells (E:T 20:1) sensitised with the indicated peptide (10 μM) in a standard 5 hour chromium release assay¹⁹. Bulk effectors generated by the addition of irradiated autologous A type LCLs¹⁹ (LCL to PBMC ratio 1:50) gave similar results to that shown above.

Two linear B cell epitopes (STNS and NNLVSGPEH) recognised by monoclonal antibodies (8G10/48²² and 8E7/55²³ respectively) were incorporated at each end of the polytope construct (FIG. 1) to follow the expression of the polytope protein. Western blotting and indirect immunofluorescence antibody staining of polytope vaccinia infected lymphoblastoid cell lines (LCLs) and the processing defective T2 cell line^{6,7} using these antibodies failed to detect polytope protein products (data not shown). Recombinant proteins expressed by vaccinia using the same P7.5 promoter are usually readily detected²⁴ implying that the polytope protein was rapidly degraded in the cytoplasm of mammalian cells. This degradation was not dependent on LMP2 and 7 since the T2 cell line does not express these proteosome associated endopeptidases^{6,7}. This phenomenon is consistent with other studies expressing truncated proteins or peptides in mammalian cells²⁵ and is likely to reflect the inability of such proteins to fold into any secondary or tertiary structures.

A glutathione S-transferase fusion expression vector containing the human polytope was constructed. The DNA fragment coding for the human polytope was excised from pBSpolytope using BamHI/HincII and cloned into the BamHI/AmaI restriction sites in pGex-3x (GST Gene Fusion System Pharmacia) to make pFuspoly. This plasmid was used to express the polytope fusion in bacteria using the standard induction protocols. An aliquot of the bacteria was lysed in loading buffer and run on a 20% SDS PAGE gel with size markers. This gel indicated that the expected protein of approximately 38 kD (the human polytope plus the GST domain (26 kD)) was being expressed in bacteria containing the plasmid. Western blotting with the two monoclonal antibodies 8G10/48 and 8E7/55 demonstrated that the fusion detected contained the human polytope which has the two linear B cell epitopes (STNS and NNLVSGPEH respectively) incorporated at each end of the polytope construct. This protein may be incorporated into liposomes or ISCOMs.

Attempts to purify the fusion protein using the GST purification employing glutathione agarose beads failed due to the lack of fusion protein in the bacterial extract supernatant. All the fusion protein precipitated with the cell debris. The protocol was not at fault since GST expressed by itself in a different bacterial culture was in the bacterial extract supernatant and could be purified easily. These data suggest the fusion protein is rapidly degraded in the bacteria unless sequestered into bacterial inclusion bodies from which purification using the GST system is difficult.

### EXAMPLE 2

Materials and Methods

Construction of a recombinant vaccinia expressing the murine polytope protein. Ten class I murine CTL epitopes from various diseases were selected so that there were two epitopes for each of H-2 Db, H-2 Kb, H-2 Kd, H-2Kk and H-2Ld which are represented in three strains of mice (see Table 2). These amino acid sequences were arranged such that each of the first 5 epitopes was restricted by a different HLA allele followed by the second group 6-10. The two groups of epitopes were converted to a DNA sequence using the universal codon usage data. These two DNA sequences were separated by an SpeI and flanked by a XbaI restriction site at the 5′ end and a AvrII site at the 3′ end. Also incorporated at the 5′ end is a BamHI restriction site, a Kozac sequence¹³ and a methionine start codon. While at the 3′ end there is a B cell epitope from Plasmodium falciparum, a stop codon and a SalI restriction site see FIGS. 4 and 5. Five 75mer oligonucleotides and a 76mer oligonucleotide overlapping by 20 base pairs, representing this 341 base pair sequence, were spliced together using. Splicing by Overlap Extension (SOEing)¹⁴ and the polymerase chain reaction (PCR). Primer dimers were made of primers 1 and 2, 3 and 4, 5 and 6 (0.4 μg of each) in 40 μl reactions containing standard 1× Pfu PCR buffer, 0.2 mM dNTPs and 1 U of Cloned Pfu DNA polymerase (hot start at 94° C.) using a Perkin Elmer 9600 PCR machine programmed with the following thermal program; 94° C. for 10 seconds, 42° C. for 20 seconds and 72° C. for 20 seconds for 5 cycles. At the end of 5 cycles the PCR programme was paused at 72° C. and 20 μl aliquots of reactions 2 and 3 were mixed (reaction 1 was left in the PCR machine) and subjected to a further 5 cycles. At cycle 10 the program was paused again and 20 μl of reaction 1 added to the combined reactions 2 and 3 and a further 5 cycles completed. The combined 40 μl sample was then gel purified on a 4% Nusieve agarose gel (FMC) and a gel slice corresponding to the correct sized fragment removed and spun through Whatmann 3MM paper. Two 20mer oligonucleotides were used to PCR amplify the full length product using the standard reaction mix as above and an annealing temperature of 50° C. and 25 cycles. The full length PCR fragment was gel purified in a 4% Nusieve agarose gel, cloned into the EcoRV site of pBluescript IIKS⁻ to make pBSMP and checked for mutations by sequencing. The DNA insert of a plasmid containing the correct sequence was excised using BamHI/SalI restriction enzymes and cloned, using the same enzymes, behind the vaccinia P7.5 early/late promoter in the plasmid shuttle vector pBCBO7¹⁵ to generate pSTMOUSEPOLY. Construction of a TK− recombinant virus was carried out using marker rescue recombination between pSTMOUSEPOLY and VV-WR-L929 using protocols described previously¹⁶. Plaque purified virus was tested for TK phenotype and for appropriate genome arrangement by Southern blotting of viral DNA¹⁷.

Vaccination of mice with recombinant murine polytope vaccinia. The recombinant vaccinia was used to vaccinate 3 mice in each of the 3 strains of mice Balb/cv, C57BL/6 and CBA. The vaccinations were I.V. 50 μl containing 5×10⁷ pfu of vaccinia and the mice were left to recover for three weeks. The TK− vaccinia was used as a negative control for each strain of mouse in this experiment.

Cytotoxic T cell assays. Splenocytes were harvested from the vaccinated mice 3 weeks post vaccination and restimulated with the appropriate peptides (1 μg/ml) in vitro¹⁶. No peptide were used for restimulations as negative controls. After 7 days of culture the restimulated bulk effectors were harvested and used in a 5 hour, ⁵¹Cr-release assays. The targets used in these assays were ConA blasts generated from each of the strains coated with one of the peptides presented by that strain. Three effector to target ratios were used 50:1, 10:1 and 2:1 the results are shown in FIG. 6.

Results

Construction of murine recombinant polytope vaccinia, The list of epitopes included in the murine polytope are listed in Table 2.

TABLE 2
CTL epitopes of the murine CTL polytope

| | | RE- | |
|---|---|---|---|
| | | STRIC- | MOUSE |
| SOURCE | SEQUENCE | TION | STRAIN |
| Influenza nuclear protein | ASNENMDAM | H-2D^{b} | C57BL/6 |
| (366-374) | | | |
| Adenovirus 5 E1A (234-243) | SGPSNTPPEI | H-2D^{b} | C57BL/6 |
| Ovalbumin (257-264) | SIINFEEKL | H-2K^{b} | C57BL/6 |
| Sendai virus nuclear protein | FAPGNYPAL | H-2K^{b} | C57BL/6 |
| (324-332) | | | |
| Influenza nuclear protein | TYQRTRALV | H-2K^{d} | Balb/c |
| (147-155) | | | |
| P. Berghei Circumsporozoite | SYIPSAEKI | H-SK^{d} | Balb/c |
| protein (249-257) | | | |
| Influenza nuclear protein | SDYEGRLI | H-2K^{k} | CBA |
| (50-58) | | | |
| Influenza NS1 (152-160) | EEGAIVGEI | H-SK^{k} | CBA |
| Murine Cytomegalovirus pp89 | YPHFMPTNL | H-2L^{d} | Balb/c |
| (168-176) | | | |
| Lymphocytic choriomeningitis | RPQASGVYM | H-SL^{d} | Balb/c |
| virus nuclear protein | | | |
| (118-126) | | | |

The construction of the polytope DNA insert is summarised in FIG. 4. The polytope sequence is shown in FIG. 5.
CTL Assays.

Each epitope in the polytope induced a primary CTL response in mice with the appropriately MHC allele. No competition between two epitopes restricted by the same allele was observed. (the high flu NP response in CBA mice given TK− controls is likely to be due to a naturally acquired influenza).

Polytope constructs containing multiple CTL epitopes from various pathogens restricted by various MHC alleles are clearly capable of generating primary CTL responses to each epitope within the polytope vaccine. This has clear application in all vaccines where CTL responses are required for protection. For instance, multiple HIV CTL epitopes might be combined in a therapeutic vaccine to foreshadow epitopes expressed by escape mutants and thereby prevent disease progression.

Murine polyepitope mice have SIINFEKL specific CTL which can kill the ovalbumin transfected cell line EG7 in vitro and in vivo.

SIINFEKL Specific CTL which Kill the EG7 Tumor Cells Demonstrated In Vitro

Spleen cells from murine vaccinia immunised mice were collected 4 weeks post vaccination and restimulated in vitro with 10 ug/ml SIINFEKL for 7 days. Effectors could not lyse the untransfected parent line EL4 but could lyse the EG7 tumour cells and EL4 cells sensitised with SIINFEKL.

Protection Against EG7 Tumour in Vivo Afforded by Murine Polytope

Mice (C57B6) were given either human polytope vaccinia (Thomsom et al., 1995) or murine polytope vaccinia (10⁷ pfu/mouse/ip) and 4 weeks later received 10⁷ EL4 or EG7 tumour cells (Moore et al., 1988. Cell 54, 777) subcutaneously (10 or 11 mice per group).

The number of mice with visable tumours (all were >1 cm diameter) at day 9 is given.

| | |
|---|---|
| Human | Murine |
| Polytope | Polytope |
| Vaccinia | Vaccinia |

| EG7 | EL4 | EG7 | EL4 |
|---|---|---|---|
| 10/10* | 10/10 | 0/11 | 10/10 |

*(Two mice had tumours <1 cm in diameter)

Protection Against MCMV

BALB/c mice were challenged with MCMV (K181 strain, 8×10 PFU, 100 μl intraperitoneally) 5 weeks after polytope vaccinia vaccination. Four days after challenge the viral titres per gram of spleen were determined the results are shown in FIG. 7 (method of Scalzo et al)¹⁷.

Evaluation of Polytope Vaccines Delivered in a DNA Plasmid.

The polytope protein described above contained a linear antibody epitope recognised by a monoclonal antibody. As described above the polytope protein could not, however, be detected in cells infected with the polytope vaccinia indicating that it is very unstable; a likely consequence of having no folding structure. It was thus considered that delivery of a polytope vaccine may be best achieved using nucleic acid vaccination technology or with an adjuvant system that protects from proteolysis (eg liposomes or ISCOMS).

The CMV promotor cassette from pCIS2.CXXNH (Eaton et al (1986) Biochemistry 25(26) p8343) was cloned into the EcoRI site of pUC8 in the same orientation as the LacZ gene to make the plasmid pDNAVacc (used as a control plasmid in the DNA vaccination experiments). This plasmid then had the murine polytope (from pBSMP) inserted into the XhoI site in the multiple cloning site to form pSTMPDV. The plasmid pRSVGM/CMVMP has fragments sourced from a number of different plasmids. The RSV promotor was excised from pRSVHygro (Long et al (1991) Hum. Immunol. 31, 229-235), the murine GM-CSF gene from pMPZen (GM-CSF) (Johnson et al (1989) EMBO 8, 441-448) and the CMV promotor cassette from pCS (Kienzie et al (1992) Arch. Virol. 124 p123-132). Into the CMV cassette was the murine polytope cloned into the SmaI site of the multiple cloning site. Both genes, murine GM-CSF and the murine polytope, use the bi-directional polyA from SV40.

Nine 6 week old female Balb/c mice were injected I.M. with 50 μg of either pDNAVacc plasmid control), pSTMPDV (murine polytope only) or pRSVGM/CMVMP (murine GM-CSF and murine polytope) in 50 μl of PBS (see next figure). They were given boosters with another 50 μg of the same plasmids at 3 weeks. At 8 weeks from the vaccination these mice were killed and their spleens removed. Splenocytes were isolated and cultured with peptide as previously described for vaccinia vaccinated animals. These bulk effectors were then used in standard ⁵¹Cr release assays against P815 cells coated with peptide corresponding to the epitopes in the murine polytope that are presented by Balb/c mice. The assay was done for 6 hours at E:T ratios of 2:1, 10:1 and 50:1.

The results of these experiments are shown in FIG. 8.

Specific CTL Activity Against Peptide Coated and Virus Infected Targets Induced by the Murine Polytope Vaccinia

Method

1. Vaccination and Effector Cell Preparation. Mice (3 per group) were vaccinated intraperitoneally (IP) with 5×10⁷ PFU vaccinia. Mice were boosted via the same route and with the same amount of vaccinia week 3. The spleens were removed 6 weeks after the initial vaccination and the splenocytes were isolated after erythrocyte lysis with ACK Buffer (0.15M N₄Cl, 1 mM KHCO₃, 0.1 mM Na₂EDTA)(Current Protocols in Immunology, Ed J E Coligan, A M Kruisbeek, D H Margulies, E M Shevach, W Strober, 1994 John Wiley and Sons Inc. USA.). 5×10⁶ splenocytes per well were peptide restimulated (1 μg/ml) in bulk T cell media (RPMI/10% Fetal Calf Serum (FCS), 2 mM Glutamine, 5×10⁻⁵M 2-Mercaptoethanol) for seven days prior to cytotoxic T lymphocyte (CTL) assay on ⁵¹chromium (⁵¹Cr) labelled target cells¹⁷. The peptides used for restimulation are given above A to J. The effectors were used against either peptide coated targets A-J, viral infected targets (A′-J′) or transfected antigen expressing targets (I′). 2. Preparation of Target Cells. Cell lines used as targets in these assays were P815 for Balb/c (H-2^{d}), EL-4 and EG7 for C57BL/6 (H-2^{b}), L929 for CBA (H-2^{k}) L929, or con A blasts prepared from the Balb/c, C57BL/6 or CBA mice, respectively. To express the required epitope for CTL killing, target cells were either pre-incubated with (i) peptide (A-J), (ii) vaccinia (B′-D′, F′-J′). or (iii) Influenza (A′, E′), or maintained as the (iv) Ovalbumin-expressing plasmid transfectant of El-4 (EG7) in the case of the SIINFEKL epitope system (I′).

(i) Peptide coated targets (A-J): Target cells were centrifuged at 1000 rpm/5 min. The supernatant was discarded to approximately 200 μg/ml and 10-20 μl of either RPMI (No peptide) or 200 ug/ml stock peptide in RPMI (peptide coated) (final concentration 10 μg/ml) was added to the cell pellet. One hundred microlitres of ⁵¹Cr was added to cell pellet and the cells were incubated at 37° C. for 1 hr. The cells were then washed twice with RPMI/10% FCS through a FCS underlayer and resuspended at 10 ⁵/ml for target cells in the CTL assay.

(ii) Vaccinia (Vacc.) infected targets (B′-D′, F′-J′): Vaccinia used for virus infected targets were the Murine Polytope (Vacc Mu PT), with the Human Polytope (Vacc Hu PT) as the negative control. Cell lines infected by vaccinia were P815 (B′-D′), L929 (F′) and EL-4 (G′-J′). The target cells were centrifuged at 1000 rpm/5 min. The supernatant was discarded to approximately 200 ul and the cells (approx. 10⁶ cells) infected with vaccinia at a multiplicity of infection (MOI) of 10:1 by adding 20 μl vaccinia (10⁹ pfu/ml) followed by incubation for 1 hr at 37° C. Five millilitres of RPMI/10% FCS was then added, cells mixed and incubated overnight at 37° C. These cells were subsequently centrifuged and supernatant discarded into camdyne. One hundred microlitres of ⁵¹Cr was added to cell pellet and the cells incubated at 37° C. for 1 hr. The cells were then washed twice with RPMI/10% FCS through a FCS underlayer and resuspended at 10⁵/ml for target cells in the CTL assay.

(iii) Influenza infected targets (A′, E′): The A/PR/8/34 strain of Influenza virus was used for the Balb/c targets (A′) and the reassortant A/Taiwan/1/86 (IVR-40) for the CBA targets (E′). Allantoic fluid was used as the negative control. Cell lines infected by influenza were P815 (A′) and L929 (E′). Target cells were centrifuged at 1000 rpm/5 min. and supernatant was discarded. Five hundred microlitres: 50 μl Influenza virus (10⁸/ml EID) or Allantoic Fluid, 50 μl ⁵¹Cr, 400 μl RPMI/1% FCS was added to the cell pellet and incubated for 1 hr at 37° C. Ten millilitres of RPMI/10% FCS was added, mixed and incubated a further 2 hr at 37° C. The cells were then washed twice with RPMI/10% FCS through a FCS underlayer and resuspended at 10⁵/ml for target cells in CTL assay.

(iv) Ovalbumin expressing targets (I′): EG7 cells are EL-4 cells transfected with an expression plasmid containing chicken ovalbumin cDNA (Moore M W, Carbone F R and Bevan B J (1988) Introduction of soluble protein into Class 1 pathway of antigen processing and presentation. Cell 54: 777-785.). These cells were maintained in RPMI/10% FCS. 20 mM Hepes, 2 mM Glutamine, 1 mM Na Pyruvate, 100 μU/ml penicillin and 100 g/ml Streptomycin. The plasmid was selected and maintained in Geneticin (G-418) at 500 μg/ml once per month. EL-4 cells with no peptide (EL4 no pep) were used as the negative control. The cells were centrifuged at 1000 rpm/5 min. and supernatant discarded to approximately 200 μl. One hundred microlitres of ⁵¹Cr was added to cell pellet and the cells incubated at 37° C. for 1 hr. The cells were then washed twice with RPMI/10% FCS through a FCS underlayer and resuspended at 10⁵/ml for target cells in the CTL assay.

3. CTL Assay. The restimulated splenocytes (5×10⁶/ml) were dispensed (100 μl) in triplicate at three Effector: Target ratios (50, 10, 2×10⁴ effector cells: 1×10⁴ target cells for the CTL assay. One hundred microlitres of target cells (10⁵/ml) were added to all effectors and control wells (Spontaneous release=100 μl media; Maximal release=100 μl 0.5% SDS/RPMI/10% FCS). Microtitre plates were centrifuged at 500 rpm for 5 min. and incubated at 37° C. for 6 hr. Plates were recentrifuged at 500 rpm/5 min. and 25 μl of supernatant was counted for ⁵¹Cr release. Percentage Specific Lysis represents averages of triplicate counts: 100×(Test cpm−Spontaneous cpm)/(Maximal cpm−Spontaneous cpm).

The results are shown in FIG. 9.

DNA Vaccination Experiment

The initial DNA vaccination experiments illustrate that the polytope can be delivered using DNA vaccination. In addition, that vaccination may be improved by the co-delivery of a cytokine gene (GM-CSF), although in this experiment the improvement is not statistically significant.

The current system is clearly sub-optimal. Likely improvements would be the use of potentially better plasmid vectors e.g. the vectors from Vical, San Diego (Sedegah M, R Hedstrom, P Hobart, S L Hoffman, 1994. Protection against malaria by immunisation with plasmid DNA encoding circumsporozoite protein. PNAS 91, 9866-9870) and the use of better delivery systems (to IM injection) employing a gene gun (Sun W H., Burkholder J K., Sun J., Culp J., Lu X G., Pugh T D., Ershler W B, Yang N S. IN VIVO CYTOKINE GENE TRANSFER BY GENE GUN REDUCES TUMOUR GROWTH IN MICE. Proceedings of the National Academy of Sciences of the United States of America. 92:2889-2893, 1995.). In addition priming against CTL epitopes usually requires CD4 T cell help¹⁷ thus the inclusion helper epitopes or proteins in the construct may improve the level and reliability of CTL priming by the murine DNA vaccine polytope.

Lack of “Original antigenic sin” or the ability of a polytope to raise immune responses to all the epitopes in a polytope when the individual has already got a response to one of the epitopes.

Introduction

Original antigenic sin is a term given to an antibody based phenomena whereby an existing antibody response to an epitope prevents the raising of an immune response to a second epitope when that epitope is present on the same protein as the first epitope (Benjamini E., Andria M. L., Estin C. D., Notron, F. L. & Leung C. Y. (1988) Studies on the clonality of the response to an epitope of a protein antigen. Randomness of activation of epitope—recognizing clones and the development of clonal dominance. J. Immunol. 141,55.). The reason for this phenomena is that large population of primed B cells specific for the first epitope bind and mop up all the available antigen before a naive B cell specific for the second antibody has a chance to bind the antigen, process it and present it to T helper cells. A similar situation might occur when an individual is vaccinated with a polytope when he/she already has a response to one of the epitopes in the polytope. The existing CTL might kill all the polytope expressing cells before any of the other epitopes can be presented to naive T cells.

Method

To test this possibility mice (Balb/c) were infected with 10⁴ pfu of Murine cytomegalovirus (MCMV) (K181 strain—Scalzo et al. 1995) and left for 5 weeks at which point strong CTL responses specific for the MCMV epitope, YPHFMPTNL, had developed (Scalzo et al. 1995—FIG. 2A). These mice were then given the murine polytope vaccinia and spleen cells assayed 10 days later for CTL specific for the three other epitopes presented by the polytope in this strain of mouse (RPQASGVYM, Lymphocytic choriomeningitis virus nuclear protein, H-2L^{d}; TYQRTRALV, influenza nuclear protein, H-2K^{d} and SYIPSAEKI, P. Berghei circumsporozoite protein, H-2 K^{d}).

Results

Responses to each of the three new epitopes was observed following polytope vaccination, illustrating that the YPHFMPTNL specific CTL did not prevent priming of CTL specific for RPQASGVYM, TYQRTRALV and SYIPSAEKI when all four epitopes are presented together in the polytope. (Control animals receiving the human polytope vaccinia instead of the murine polytope vaccinia, showed only YPHFMPTNL specific CTL).

This series of experiments illustrate that if a polytope was, for instance, designed to cover a variety of different diseases, an individual receiving such polytope vaccine, but who had already been exposed to one of the target diseases would still be immunised against the remaining CTL epitopes in the polytope.

As will be apparent to those skilled in the art the present inventors have shown that the natural flanking sequences of CTL epitopes are not required for class I processing, that is each epitope within the polyepitope protein was always efficiently processed and presented to appropriate CTL clones by autologous polyepitope vaccinia infected target cells. It will be apparent to those skilled in the art that the polytopes may include sequences not naturally found to flank the epitopes.

As discussed above the present invention can be used with a range of epitopes. A range of epitopes are now available on an Internet address which is described in Brusic et al Nucleic Acids Research, 1994, 22; 3663-5.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Elliott, T., Smith, M., Driscoll, P. & McMichael, A. Curr. Biol. 3, 854 (1993).
2. Goldberg, A. L. and Kennith, L. R. Nature 357, 375-379 (1992).
3. Michalek, M. T., Grant, E. P., Gramm, C., Goldberg, A. L. & Rock, K. L. Nature 363, 552-554 (1993).
4. Driscoll, J., Brown, M. G., Finely, D. & Monaco, J. J. Nature 365, 262-264 (1993).
5. Gaczynska M., Rock, K. L. & Goldberg, A. L. Nature 365, 264-267 (1993).
6. Arnold, D. et al., Nature 360, 171-174 (1992)
7. Momburg, F. et al., Nature 360, 174-177 (1992)
8. Oldstone, M. B. A. et al. J. Virol. 67, 4372-4378 (1993).
9. Whitton, J. L., Sheng, N., Oldstone, M. B. A. & McKee, T. A. J. Virol 67, 348-352 (1993).
10. Khanna, R. et al. J. Exp. Med. 176, 169-176 (1992).
11. Johnson, R. P., Trocha, A., Buchanan, T. M. & Walker, B. D. J. Virol 67, 438-445 (1993).
12. Ulmer, J. B. et al. Science 259, 1745-1748 (1993).
13. Kozak, M. Cell 44, 283-292 (1986).
14. Ho, S. N., Hunt, H. D., Horton, R. M., Pullen, J. K. & Pease, L. R. Gene 77, 51-59 (1989.).
15. Andrew, M. E. et al. J. Virol. 61, 1954-1060 (1987).
16. Boyle, D. B., Coupar, B. E. H. & Both, G. W. Gene 35, 169-177 (1985).
17. A. A. Scalzo, S. Elliot, J. Cox, J. Cardner, D. J. Moss and A. Suhrbier. 1994. Induction of protective cytotoxic T cells to murine cytomegalovirus using a nonapeptide and a human compatable adjuvant (Montanide ISA 720). Journal of Virology 65: 1306-1309.
18. Moss, D. J., Burrows, S. R., Khanna, R., Misko, I. S. & Sculley, T. B., Seminars in Immunology r, 97-104 (1992).
19. Burrows, S. R. et al, J. Gen. Virol. (Accepted) (1994).
20. Levitsky, V. et al, J. Exp. Med. 176, 1297-1305 (1994)
21. Khanna, R., Jacob, C. A., Burrows, S. R. & Moss D. J., J. Immunol. Meth. 164, 41-49 (1993).
22. Epping, R. J. et al, Mol. Biochem. Parasitol. 28, 1-10 (1988).
23. Kara, U. et al, Mol. Biochem. Parasitol. 38, 19-24 (1990).
24. Khanna, R. et al, Immunol. 74, 504-510 (1991).
25. Eisenlohr, L. C., Yewdell, D. W. & Bennink, J. Exp. Med. 175, 481-487 (1992)

1-13. (canceled)

14. A method for producing a nucleic acid polytope construct including the steps of:
(i) constructing a polynucleotide comprising a nucleotide sequence encoding a plurality of cytotoxic T lymphocyte (CTL) epitopes of at least one pathogen, wherein each CTL epitope is substantially free of sequences naturally found to flank said CTL epitope; and
(ii) operably linking the polynucleotide in (i) to a promoter for expression of the polynucleotide in an animal to thereby produce the nucleic acid polytope construct.

15. The method of claim 1 wherein each CTL epitope does not include any sequences naturally found to flank each CTL epitope.

16. The method of claim 2 wherein at least two of the CTL epitopes are contiguous.

17. The method of claim 3 wherein each adjacent CTL epitope is contiguous.

18. The method of claim 1 wherein said polynucleotide encodes at least three CTL epitopes.

19. The method of claim 1 wherein said polynucleotide encodes four CTL epitopes.

20. The method of claim 1 wherein said polynucleotide encodes nine CTL epitopes.

21. The method of claim 1 wherein said polynucleotide encodes ten CTL epitopes.

22. The method of claim 1 wherein said polytope construct comprises a vector.

23. The method of claim 9 wherein said vector is selected from the group consisting of a vaccine vector, avipox vector, bacterial vector, virus-like particle (VLP) and rhabdovirus vector.

24. The method of claim 1 wherein said polynucleotide comprises a nucleotide sequence encoding CTL epitopes of a plurality of pathogens.

25. The method of claim 1 wherein said pathogen is selected from the group consisting of Epstein Barr Virus, Influenza Virus, Cytomegalovirus and Adenovirus.

26. The method of claim 1 wherein said polynucleotide comprises a nucleotide sequence encoding at least one CTL epitope derived from a tumor protein.

27. The method of claim 1 wherein said polynucleotide construct further comprises a nucleic acid encoding a T helper cell epitope, a B cell epitope or a toxin.

28. The method of claim 1 wherein at least two of the encoded CTL epitopes are restricted by a same HLA allele.

29. The method of claim 1 wherein at least two of the encoded CTL epitopes are restricted by a different HLA allele.
